(19) United States
(12) Patent Application Publication
(10) Pub. No.: US 2017/0260251 A1
(43) Pub. Date: Sep. 14, 2017

(54) ECOTIN VARIANTS

(71) Applicant: Ansun Biopharma, Inc., San Diego, CA (US)

(72) Inventor: Fang Fang, San Diego, CA (US)

(21) Appl. No.: 15/514,044

(22) PCT Filed: Oct. 1, 2015
(86) PCT No.: PCT/US15/53610

§ 371 Date: Mar. 24, 2017

Related U.S. Application Data
(60) Provisional application No. 62/058,639, filed on Oct. 1, 2014.

(51) Int. Cl. C07K 14/81

(52) U.S. Cl. CPC C07K 14/811; A61K 38/00

(57) ABSTRACT

Ecotin variants and their use in treating viral hemorrhagic fever are described. Described herein are methods for treating systemic inflammatory response syndrome or viral hemorrahagic fever by administering an ecotin polypeptide. Described herein is a polypeptide comprising the amino acid sequence of any of SEQ ID NOs: 2-9 and 11-18. Also described: is a polypeptide comprising the amino acid sequence of any of SEQ ID NO: 11-18 preceded by a methionine; a polypeptide comprising the amino acid sequence of any of SEQ ID NO: 11 -18 with up to 5 single amino acid changes or deletions provided that the polypeptide does not comprise the amino acid sequence of SEQ ID NO: 10.

### BACKGROUND

Viral hemorrhagic fever (VHF) refers to a clinical illness associated with fever and a bleeding diathesis caused by a virus that belongs to one of four distinct families of enveloped, negative-sense, single-stranded RNA viruses: Filoviridae, Bunyaviridae, Flaviviridae, and Arenaviridae. A number of viruses in these four families are on the Category A biothreat list because they may cause high morbidity and mortality and are highly infectious by aerosol dissemination [1]. These viruses cause a similar spectrum of illness with similar underlying pathophysiology [2, 3]. Following an incubation period of 4-10 days, patients with VHF abruptly develop fever accompanied by prominent constitutional symptoms such as prostration, dehydration, myalgia and general malaise. As disease progresses, patients develop clinical signs of bleeding, such as petechial hemorrhage, maculopapular rash, accompanied by disturbance of coagulation. During terminal phase of the disease, fatal cases develop disseminated intravascular coagulation (DIC), gross hemorrhage, hypotension, multi-organ failure, and shock.

Patients with severe VHF usually die from a terminal clinical course that is generally indistinguishable from systemic inflammatory response syndrome (SIRS), also referred to as sepsis, which is the common sequela of severe bacterial and viral infections. Some VHF viruses are particularly prone to cause SIRS; they include Ebola virus (EBOV) and Marburg Virus (MARV) in Filoviridae, Rift Valley Fever virus (RVFV) and Hantaviruses in Bunyaviridae, and Dengue virus in Flaviviridae [4, 5].

### SUMMARY

Described herein are methods for treating systemic inflammatory response syndrome or viral hemorrahagic fever by administering an ecotin polypeptide.

Described herein is a polypeptide comprising the amino acid sequence of any of SEQ ID NOs: 2-9 and 11-18. Also described: is a polypeptide comprising the amino acid sequence of any of SEQ ID NO:11-18 preceded by a methionine; a polypeptide comprising the amino acid sequence of any of SEQ ID NO:11-18 with up to 5 single amino acid changes or deletions provided that the polypeptide does not comprise the amino acid sequence of SEQ ID NO:10; a polypeptide having up to 3 single amino acid changes provided that the polypeptide does not comprise the amino acid sequence of SEQ ID NO:10; a polypeptide having up to 3 single amino acid changes provided that the polypeptide does not comprise the amino acid sequence of SEQ ID NO:10; a polypeptide having up to 2 single amino acid changes provided that the polypeptide does not comprise the amino acid sequence of SEQ ID NO:10; a polypeptide no more that one amino acid change provided that the polypeptide does not comprise the amino acid sequence of SEQ ID NO:10; a polypeptide comprising the amino acid sequence of any of SEQ ID NO:2-9 and 11-18 with up to 5 single amino acid changes or deletions provided that the polypeptide does not comprise the amino acid sequence of SEQ ID NO:1 or 10; a polypeptide having up to 3 single amino acid changes provided that the polypeptide does not comprise the amino acid sequence of SEQ ID NO:1 or 10; a polypeptide having up to 3 single amino acid changes provided that the polypeptide does not comprise the amino acid sequence of SEQ ID NO:1 or 10; a polypeptide having up to 2 single amino acid changes provided that the polypeptide does not comprise the amino acid sequence of SEQ ID NO:1 or 10; a polypeptide having no more that one amino acid change provided that the polypeptide does not comprise the amino acid sequence of SEQ ID NO:1 or 10; and any such polypeptide preceded by a methionine.

Further described is a pharmaceutical composition comprising a polypeptide described herein and a pharmaceutically acceptable carrier or excipient. Also discloses is method for treating a patient infected with a microorganism that causes viral hemorrhagic fever, the method comprising administering the pharmaceutical composition or polypeptide described herein. In various embodiments: the patient is infected with a virus from a family selected from the group consisting of: Filoviridae, Bunyaviridae, Flaviviridae, and Arenaviridae; and the patient is infected with a virus selected from Ebola virus (EBOV), Marburg Virus (MARV), Rift Valley Fever virus (RVFV), Hantaviruses, and Dengue virus.

Also described is a nucleic acid molecule comprising a sequence encoding the polypeptide described herein as well as such a nucleic acid molecule further comprising an expression control sequence operably linked to the sequence encoding the polypeptide. Also describe is a recombinant cell comprising a nucleic acid molecule described herein and a method of producing a polypeptide comprising culturing a recombinant cell of described herein under conditions suitable for expressing the encoded polypeptide and isolating the encoded polypeptide from the recombinant cells.

The details of one or more embodiments of the invention are set forth in the accompanying drawings and the description below. Other features, objects, and advantages of the invention will be apparent from the description and drawings, and from the claims.

### DRAWINGS

FIG. 1A-B. Effect of NB109 and NB101 on human blood coagulation in vitro. Increasing concentrations of NB109 and NB101 were preincubated with the human plasma samples for 15 min at 37° C. (A) PT and (B) APTT assays were performed using an ACL100 automated coagulometer using standard reagents. Prolongation of clotting time was calculated as (value in candidate treated sample)/(value in control sample) and was plotted against drug concentrations. 1.2-fold and 1.5-fold over the control in clotting times for PT and for APTT respectively are indicated with dashed lines. Error bars represent SEM, n=3 plasmas from three different donors.

FIG. 2. Effect of NB101 and NB109 in mice endotoxemia model. BALB/c female mice (N=5) were subjected to two injections of lipopolysaccharide (LPS) as a model for DIC. A 5 μg/mouse priming dose of LPS was injected into the footpad at t=0 hr, followed 24 hours later by an intraperitoneal elicitation dose of 400 μg/mouse. NB101, NB109 or PBS was administered 1 hr prior to the elicitation. Mice were monitored for survival on an hourly basis for up to 70 hours post-elicitation. Graphpad Prism 4.2 was used to assess statistical differences in survival curves by the Kaplan-Meier Log Rank test. Asterisks indicate significant difference between NB101 and PBS as well as NB142 and PBS *p<0.05, ***p<0.0001.

FIG. 3. Effect of NB109 on animal survival in the CLP model. CLP surgery was performed on mice. NB109 treatment was given subcutaneously 18hr before CLP (pre-loading), and twice daily follow-up. Group 2 received 60 mg/kg NB109 for pre-loading, and 40 mg/kg for follow-up. Group 3 received 30 mg/kg NB109 for pre-loading, and 20 mg/kg for follow-up. Fluid resuscitation was performed lml daily for 5 days by subcutaneous injection. Survival was observed every l2hr.

FIG. 4A-B. Effect of NB101 and NB109 in EBOV infection in guinea pigs. On day 0, strain 13 Guinea pigs (N=2) were infected by subcutaneous injection with 1000 pfu of EBOV. Compound leads were administered by i.p. injection, once a day for 7 days initiated 24 hours post-infection. Survival and body weights were monitored daily. Graphpad Prism 4.2 was used to assess statistical differences in survival curves by the Kaplan-Meier Log Rank test (*p<0.05).

FIG. 5. Coagulation parameters in mice treated with NB109. BALB/c mice given single i.p. dose of NB109. At 4, 8 and 24 hrs post dosing, PT and aPTT were analyzed. Average and standard deviation from 3-4 mice per group is presented at each time point. *: single data point. **: >180 second.

FIG. 6. Plasma concentrations of NB-109 in mice via different delivery routes. NB109 was administered to mice (n=3) at 72 mg/kg. by intravenous injection (i.v.), intraperitoneal injection (i.p.), and subcutaneous injection (s.c.). Blood samples were taken at different time points. Data shown at Mean±SD.

FIG. 7. Plasma concentrations of NB109 and coagulation parameters in guinea pigs following single dose administration. Mean±SD, n=3

FIG. 8. Plasma Concentrations of NB109 and Coagulation Parameters in Guinea Pigs Following Single Dose Administration Blood samples collected 24 hr after each dose, and 96 hr after the last dose. Mean±SD, n=3.

FIG. 9. Effect in mice LPS model. BALB/c mice (N=10) received a 40 μg priming dose of LPS injected into the foot pad at t=0 hr, followed 24 hours later by an intraperitoneal (i.p.) elicitation dose of 400 μg of LPS. One hour prior to the elicitation dose, mice were treated with 45 mg/kg of NB101, NB109 or NB142 delivered i.p. At 2, 4 and 6 hours post-elicitation animals were bled for plasma cytokine levels. Graphpad Prism 4.2 was used to assess statistical differences in survival curves by the Kaplan-Meier Log Rank test. Asterisks indicate significant difference between NB101 and PBS as well as NB142 and PBS *p<0.05, ***p<0.0001.

FIG. 10A-C. Effect of NB109 and NB142 on animal survival in poly(I:C) challenged mice. 10 week old female BALB/c mice were randomized into vehicle and drug treatment groups (n=6). 200 ug of Poly (I:C) (polyinosinic: polycytidylic acid) was administrated i.p. twice a day, from day 0 to day 3. NB109 or NB142 treatment was given once a day, i.p., at 45 mg/kg/day, initiated on day 0, day 1, or day 2 as indicated in the figures.

FIG. 11. Effect of NB101, NB109, and NB142 on cytokines and D-dimer in poly(I:C) challenged mice. BALB/c were injected i.p. of 45 mg/kg NB101, NB109, NB142, or vehicle at 1 hr prior to poly(I:C) challenge. At time zero (t=0hr), 200 ug of Poly (I:C) or PBS per mouse was injected.

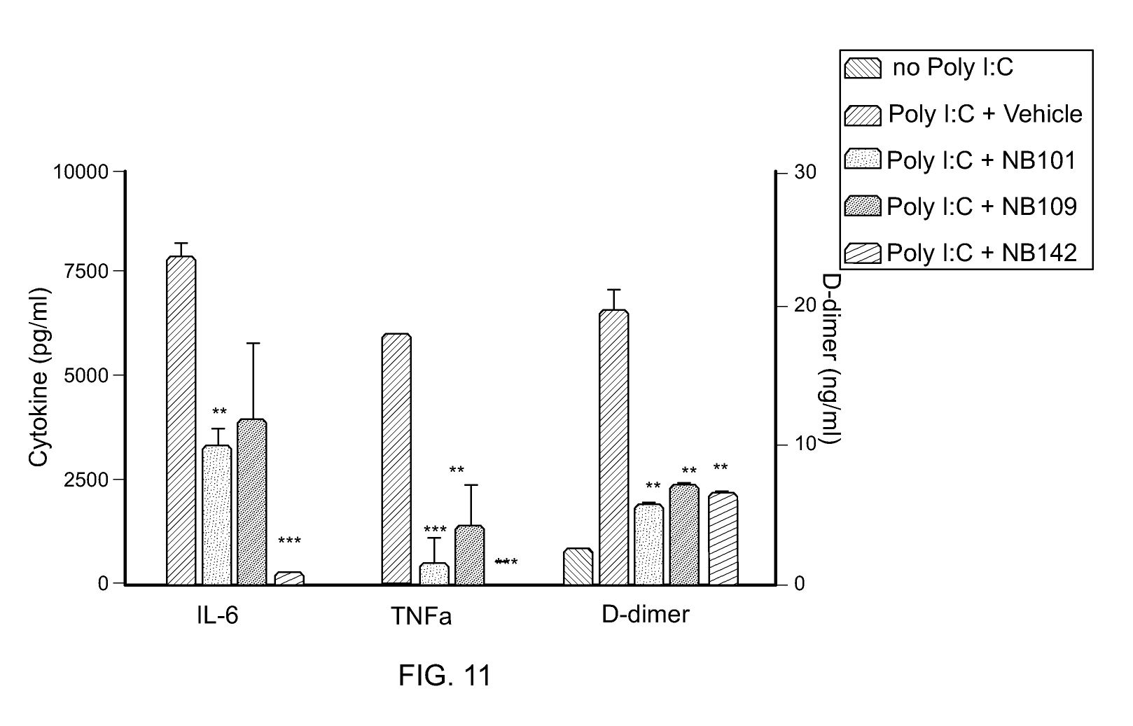

FIG. 12. Effect of NB142 and NB109 in EBOV infection in guinea pigs. On day 0 strain 13 Guinea pigs (N=3) were infected by subcutaneous injection with 1000 pfu of EBOV. Lead compounds were administered by i.p. injection, once a day for 7 days initiated 24 hours post-infection. Survival and body weights were monitored daily. Graphpad Prism 4.2 was used to assess statistical differences in survival curves by the Kaplan-Meier Log Rank test (*p<0.05)

FIG. 13. Pharmacodynamics of candidates.

BALB/c female mice (N=15) were subjected to two injections of LPS. A 5 μg/mouse priming dose of LPS was injected into the footpad at t=0 hr, followed 24 hours later by an intraperitoneal elicitation dose of 400 μg/mouse. A single dose of NB101, NB109, or NB142 at 45 mg/kg or PBS was administered 1 hr prior to the elicitation. aPTT & PT measurements were taken at indicated time points post treatment.

FIG. 14. NB109 production process flow diagram.

### DETAILED DESCRIPTION

Described below are studies on wild type Ecotin (NB101; SEQ ID NO:1) and an Ecotin variant (NB109; SEQ ID NO:2). NB109 differs from Ecotin in one amino acid residue, M84R, at the P1 position of the so-called reactive center loop (“RCL”; amino acids 82-88; amino acid number of mutations refers to the mature ectotin sequence, i.e., SEQ ID NO:1 lacking the first 20 amino acids (MKTILPAVLFAAFATTSAWA; SEQ ID NO:19) as shown in SEQ ID NO:10).

NB101 is a broad-spectrum protease inhibitor targeting serine elasase (also called neutrophil elastase (NE) or granulocyte elastase (GE)) coagulation factors (Xa, XIIa, VIIa), and kallikrein (Table 1). In addition to its potential anti-inflammatory function via NE inhibition, NB101 directly targets two components of the “SIRS triangle”; coagulation and kallikrein. However, NB101 does not inhibit fibrinolysis. Thus, all potential point mutations at the P1 position of the RCL were screened resulting in NB109. Distinct from NB101, NB109 inhibits plasmin and thrombin. As a result, it directly targets all three components of the “SIRS triangle”.

TABLE 1
Inhibition Constant Ki (nM)* of NB101 and NB109

| | | Ki (nM) | | | | | | | |
|---|---|---|---|---|---|---|---|---|---|
| Lead | Mutation | Plasmid | Kallikrein | XIIa | Thrombin | Xa | IXa | XIa | VIIa |
| NB101 | wt | DNI | 0.07/- | 0.09/- | DNI | <0.02/0.2 | 27/1.9 | 0.4/- | 0.4/- |
| NB109 | M84R | 0.3/0.2 | 0.1/0.2 | 0.2/- | 0.6/0.8 | 0.02/0.07 | 1.2/0.4 | 0.1/- | 1.1/- |

*Ki against the human and mice proteases are shown as “human/mice”.
DNI: do not inhibit.
“-”: data unavailable.

NB109 shares the chemical and physical properties with Ecotin. NB109 has an equivalent number of negatively charged residues (Asp+Glu) and positively charged residues (Arg +Lys), and the calculated pI is 6.85 [61]. One unit of compound activity is defined as the amount of compound required to inhibit 50% trypsin under the standard assay conditions. Based on this definition, NB109 has a specific activity of 1×10⁵ unit/mg, which is equivalent to NB101.

Anti-Coagulation Activity in Human Plasma

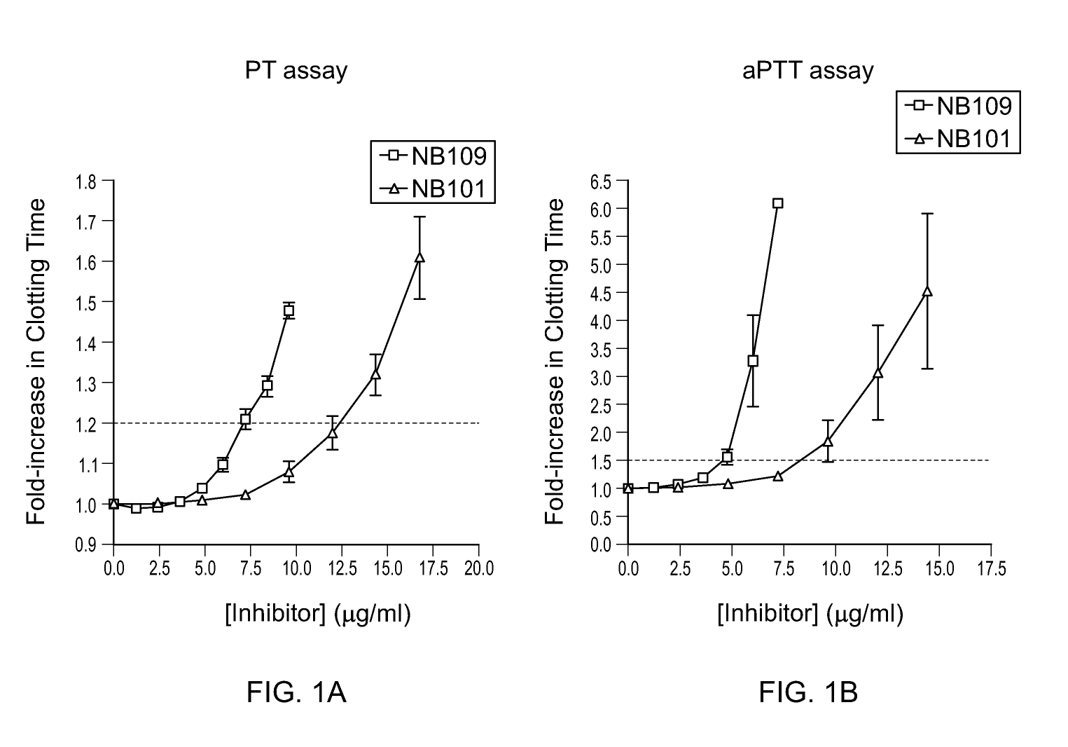

NB101 and NB109 were tested to determine their ability to inhibit blood coagulation, in particular the intrinsic pathway of blood coagulation via inhibition of inflammation and kallikrein-kinin system. The agents were test on human blood coagulation in vitro by performing PT (prothrombin time; extrinsic coagulation pathway) and aPTT (activated partial thromboplastin time; intrinsic coagulation pathway) assays. Both molecules exhibited a potent dose-dependant anti-coagulation effect, and NB109 was approximately 2 times more potent than NB101 (FIG. 1), probably due to its activity against thrombin. In addition, both NB109 and NB101 exhibited stronger inhibition (roughly two fold) towards the intrinsic coagulation pathway (as measured by aPTT) than the extrinsic pathway (measured by PT) (FIG. 1).

It is important to note that PT and aPTT elevations are expected pharmacological effects of the candidates. PT or APTT elevation per se does not signify spontaneous bleeding as an adverse effect. Spontaneous bleeding tendency is associated with uninhibited fibrinolysis and increased vascular permeability [62]. NB101 and NB109 may have a reduced risk of spontaneous bleeding because they inhibit either vascular hyper-permeability or both vascular hyper-permeability and fibrinolysis.

In Vivo Efficacy Against SIRS

NB101 and NB109 were tested in the murine endotoxemia model, which is a lethal shock model induced by two consecutive systemic exposures of endotoxin (LPS) administered 24 hr apart. Pathophysiologically, this model is characterized by inflammation, hemorrhage, tissue necrosis, and DIC [63].

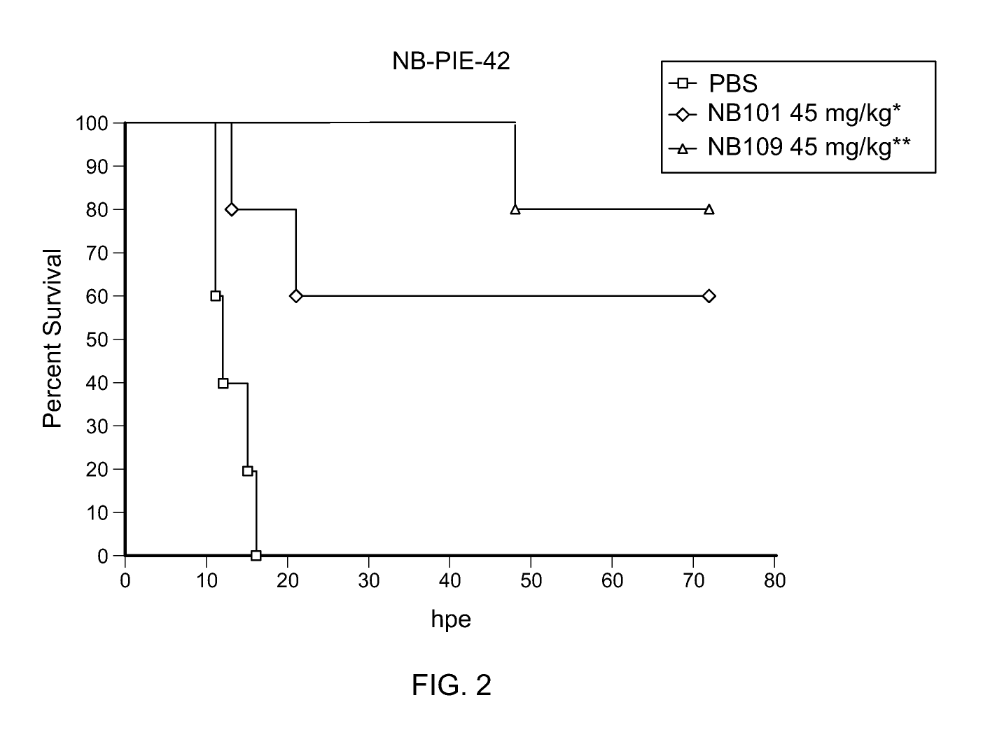

The vehicle-treated mice all suffered a rapid death within one day of LPS challenge, but treatment with NB101 and NB109 had significant survival benefit (FIG. 2). In this study, NB101 and NB109 all increased animal survival in a similar manner, and they both compared very favorably against the current standard anti-DIC treatment, low molecular weight heparin (LMWH). LMWH given twice before the LPS elicitation only improved 30-hr survival of the treated mice by 25% (50% survival in the treated group and 25% survival in the control group) [64].

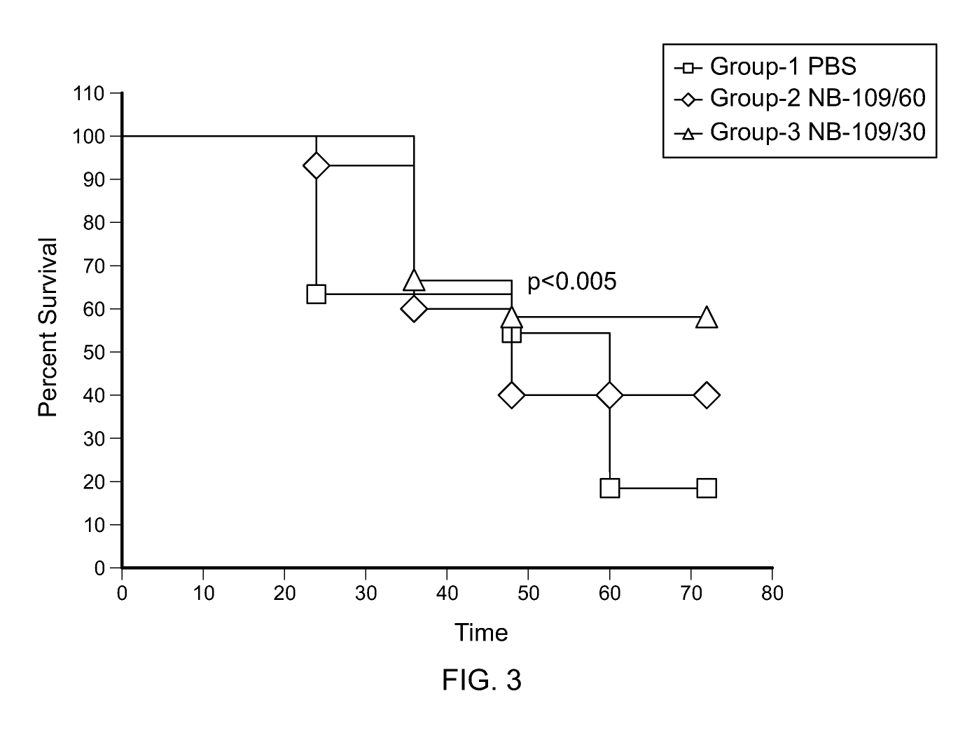

Cecal ligation and puncture (CLP) is another commonly used animal model of SIRS. In the CLP model, SIRS is produced by peritonitis following intestinal injury and infection by multiple bacteria that normally reside in the intestines. It is considered to better mimic the natural cause of sepsis [65]. In a preliminary study, NB109 achieved significant (p<0.005) survival advantage in the CLP model (FIG. 3).

In Vivo Efficacy Against VHF

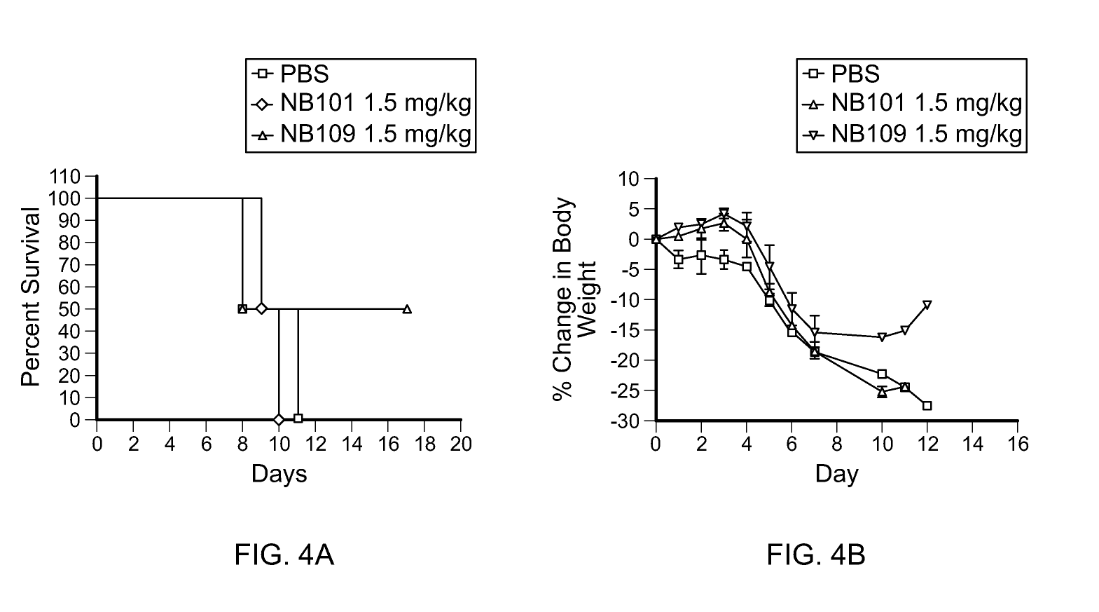

NB101 and NB109 were evaluated in guinea pigs infected with Zaire strain of EBOV. The vehicle-treated animals invariably died by Day 9. NB101 and NB109 treatment was initiated at 24 hr post infection, and was given by intraperitoneal injections once a day for 7 days. While NB101 did not affect animal survival or body weight loss, NB109 achieved 50% survival and rescued the surviving animal from fatal body weight loss (FIG. 4). This result provides proof-of-concept. Together, the in vitro and in vivo findings indicate that NB109 and NB101 are potentially potent candidates as anti-SIRS and anti-VHF compounds and pharmaceutical formulations.

Safety & PK Studies—Effect on Primary Cells

NB109 was incubated with a collection of human primary cells, including primary human renal proximal tubule cells, renal cortical epithelial cells, lung vascular endothelial cells, or hepatocytes, as well as cells lines, A549 and BEAS-2B, at up to 250 μM. Over 4-day incubation, cytotoxicity was evaluated using the MTS assay. NB109 did not cause cytotoxicity and had no effect on viability of the cells.

Effect on Hemolysis

NB109 was examined for indirect hemolysis via activation of complements, or direct hemolysis. As a positive control for the complement-mediated hemolysis, species specific antibodies against red blood cells (RBC) were used to activate the classical complement pathway and initiate the signaling cascade leading to the lysis of the RBC. For evaluating direct hemolytic activity of NB109, the RBC were washed to remove any complement proteins, and then resuspended with heat-inactivated plasma or serum containing NB109. In the human blood, NB109 did not elicit hemolytic reactions, neither direct nor complement mediated, at concentrations up to 1 mg/ml.

Systemic Safety of NB109 Treatment in Mice Safety and tolerability of NB109 systemic treatment in mice was evaluated in 5 groups of 16 BALB/c mice. Each of the four groups received one intraperitoneal injection of NB109 at 5, 15, 45, and 90 mg/kg, respectively; the fifth received PBS. Mice were sacrificed at 4 hr and 24 hr post dosing and subjected to necropsy, coagulation analysis, and clinical chemistry.

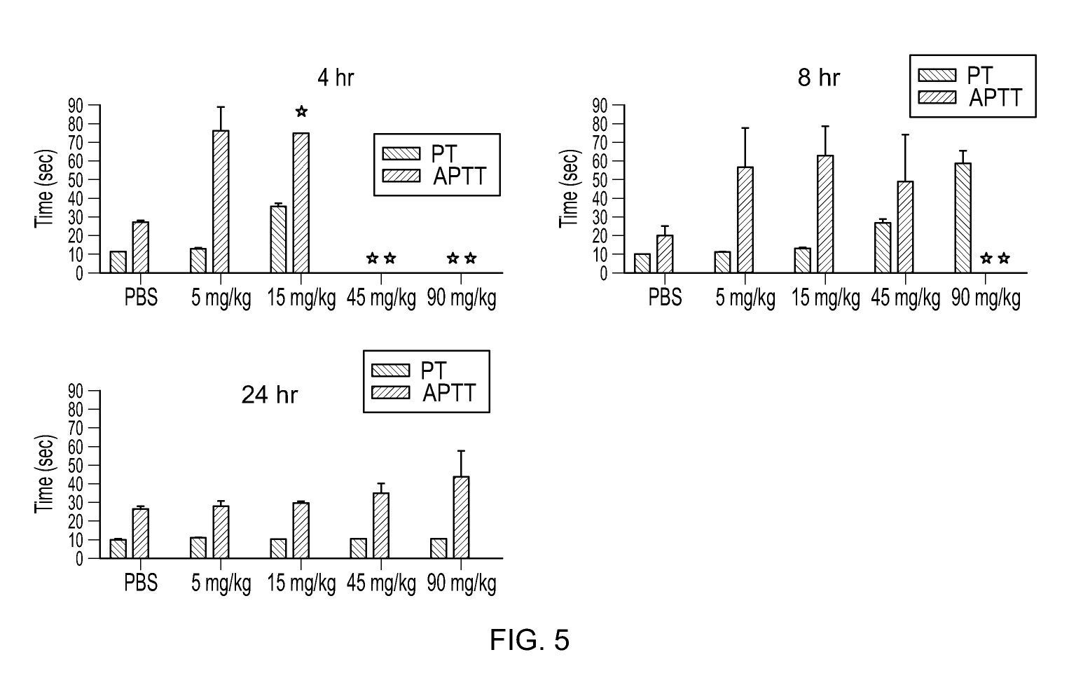

Upon necropsy, all animals appeared to be normal without signs of hemorrhage. As expected, coagulation parameters were affected in a dose-dependent manner; the effects peaked at 4 hr post treatment and returned to the baseline by 24 hr post treatment (FIG. 5), which indicates that NB109 was cleared from the blood within 24 hours. Consistent with what was observed in the human blood, aPTT was more sensitive to NB109 and the effect was observed at 5 mg/kg whereas PT was not affected until 15 mg/kg. PT returned to the baseline level before aPTT did. It should be noted that elevations in PT and aPTT are pharmacological effects and are not considered adverse effects.

Repeated Dose Toxicity Study in Guinea Pigs

NB109 was given to Hartley guinea pigs by intraperitoneal administration at doses of 0.1, 0.5, 1.5, and 5 mg/kg/day for 7 days. Safety parameters included clinical signs, serum chemistry, coagulation times, and necropsy.

All animals survived NB109 treatment, and all clinical observations for NB109-treated animals were normal throughout the course of the study. There was no significant difference in body weight change between the groups, and all groups showed significant weight gain (19-23% by the end of the study). Necropsy of all NB109-treated animals was unremarkable.

There was a trend of mild and transient elevation of Creatine phosphokinase (CPK) at ≧1.5 mg/kg on Day 2, but the values returned to the normal range by Day 7. A mild elevation of AST was seen on Day 14 at ≧1.5 mg/kg, but other liver enzymes and bilirubin were normal. All other clinical laboratory parameters were within the normal range. No changes in coagulation parameters were observed at doses 1.5 mg/kg and below (Note that the guinea pig has reduced FVII levels, thus a longer PT than other species). At 5.0 mg/kg, elevated PT and aPTT values were observed starting at eight hours after the first dose and continuing on through eight hours after the last dose on Day 7. All PT and aPTT values returned to normal by Day 14.

Preliminary Pharmacokinetic Analysis

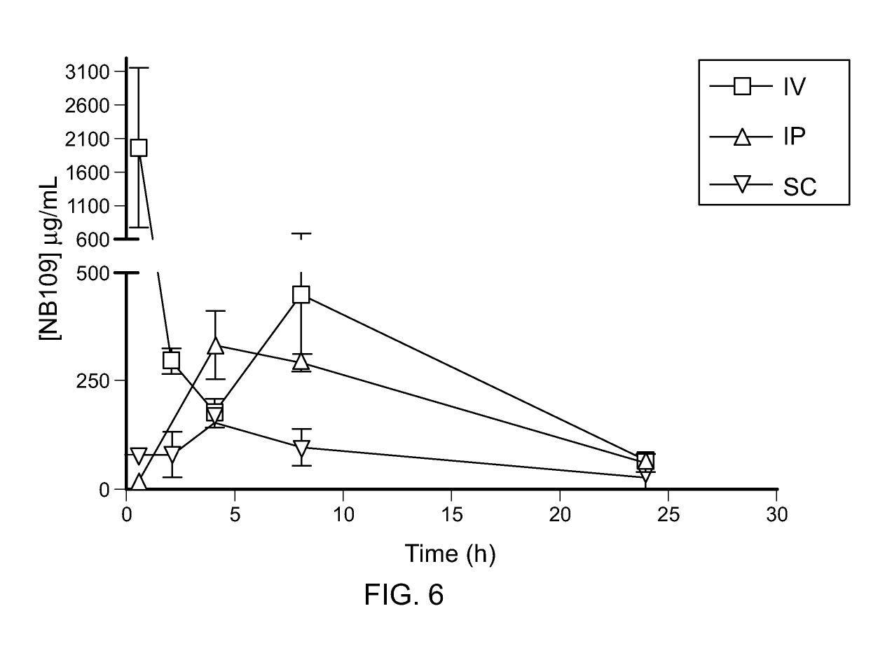

A preliminary pharmacokinetic (PK) study was conducted in mice in which NB109 was administered by different routes. The data are illustrated in FIG. 6. Initial plasma concentrations were much higher with IV administration relative to IP or SC injection. Intraperitoneal injection resulted in considerably higher concentrations than did the same dose by SC injection, meaning that the bioavailability of NB109 by the SC route would be less than ideal. Given the variability of the plasma concentration data with IV administration, it was not possible to provide any estimates of PK exposure. However, the plasma concentration for the IP route was amenable to pharmacokinetic modeling (WinNonlin software, Cary, N.C.). The half-life of elimination (t½) of NB109 by the IP route was 7.6 hr.

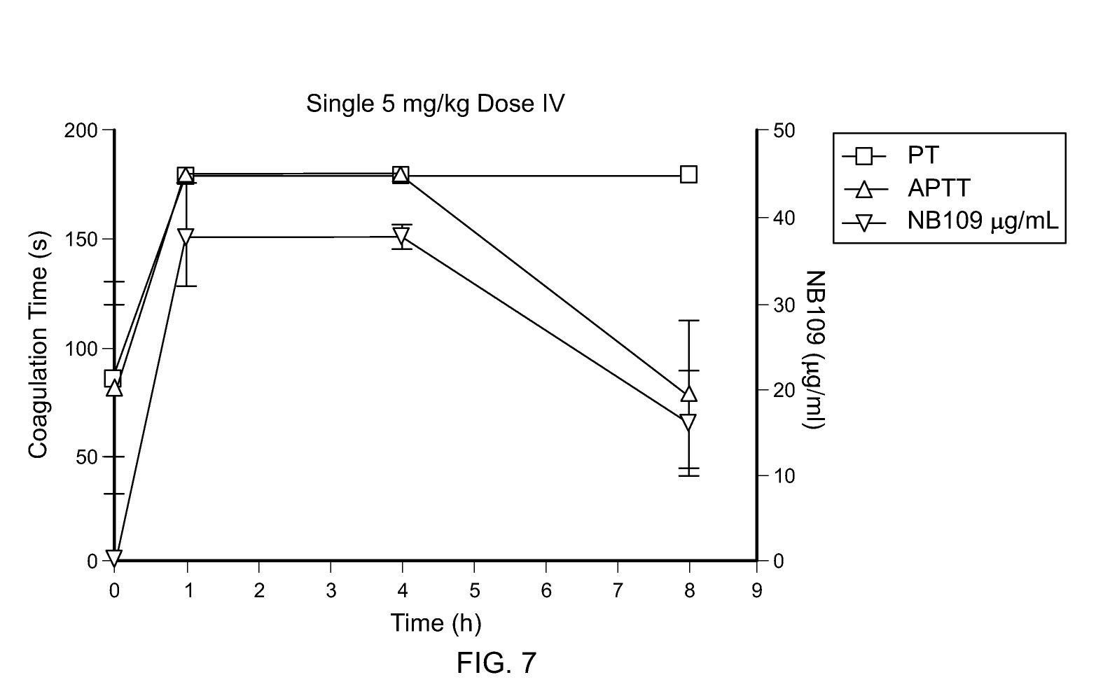

A study of NB109 was conducted in guinea pigs to evaluate the relationship between plasma concentrations of drug and blood coagulation parameters following single and repeated dose administration. NB109 was administered IV to Hartley guinea pigs (n=3) at a dose of 5 mg/kg. There was an excellent correlation between plasma levels of NB109 and prolonged aPTT following a single IV dose (FIG. 7). While the aPTT closely mirrored the plasma levels of drug which had almost returned to background levels by 8 hr post-injection, the PT remained prolonged at that time-point.

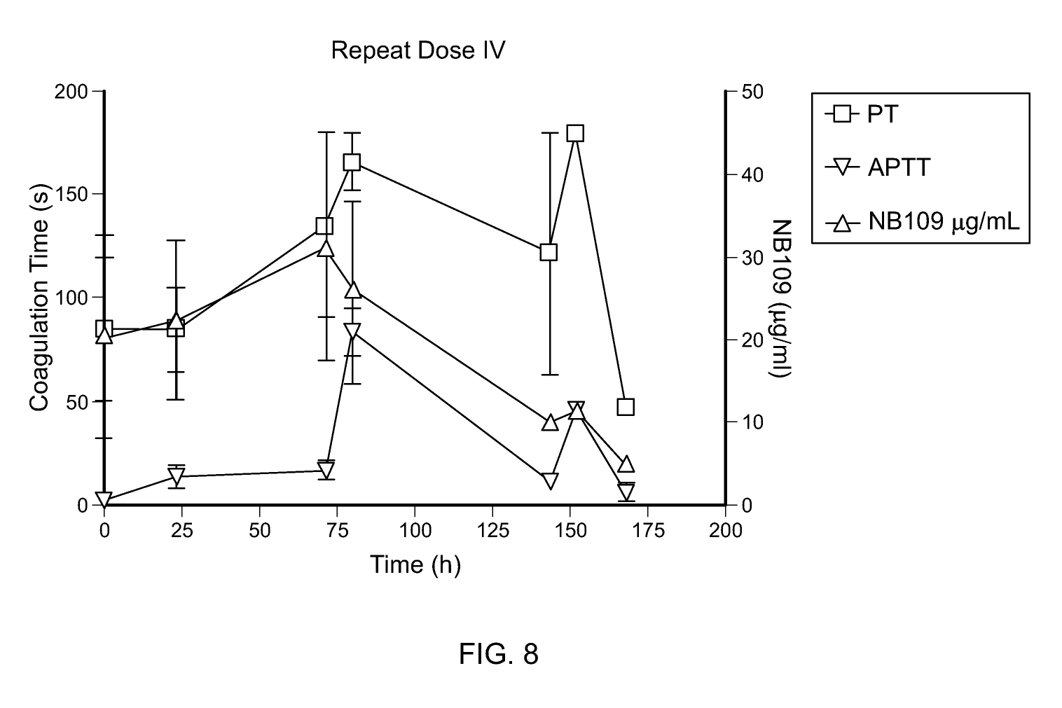

Repeated dosing was conducted again with a dose of 5 mg/kg daily for 5 days. The plasma drug levels appeared to increase slightly following the third dose, however the variability in data make any conclusions on drug accumulation difficult to determine (FIG. 8). There was a very good correlation between blood coagulation parameters with plasma NB109. By day 7 (96 hr post-dosing) all parameters had returned to baseline.

Additional Ecotin Variants

The constructs shown in Tables 2 and 2A were developed and tested and described further below. The amino acid sequence for the constructs are shown in Table 3 as SEQ ID Nos. 1-9.

TABLE 2
Inhibition Constant Ki (nM)* of Peptides

| | | Ki (nM) | | | | | | | |
|---|---|---|---|---|---|---|---|---|---|
| Lead | Mutation | Plasmin | Kallikrein | XIIa | Thrombin | Xa | IXa | XIa | VIIa |

Preliminary Candidates

| | | | | | | | | | |
|---|---|---|---|---|---|---|---|---|---|
| NB101 | wt | DNI | 0.07/- | 0.09/- | DNI | <0.02/0.2 | 27/1.9 | 0.4/- | 0.4/- |
| NB109 | M84R | 0.3/0.2 | 0.1/0.2 | 0.2/- | 0.6/0.8 | 0.02/0.07 | 1.2/0.4 | 0.1/- | 1.1/- |

Potentially Optimized Candidates

| | | | | | | | | | |
|---|---|---|---|---|---|---|---|---|---|
| NB142 | M84R/ | 3.4/0.3 | 0.07/0.1 | DNI/- | DNI | 0.04/0.09 | 58/0.8 | 0.2/- | 0.2/- |
| | V81R | | | | | | | | |
| NB137 | M84R/ | 2.9/0.3 | 0.04/0.3 | DNI/- | 1.8/7.5 | 0.4/0.1 | 0.8/0.5 | 0.07/- | 0.4/- |
| | K76I | | | | | | | | |
| NB147 | M84R/ | 4.8/2.3 | 0.2/0.30 | 9.5/- | DNI/DNI | <0.02/4.07 | DNI/DNI | 0.9/- | 0.7/- |
| | R54D | | | | | | | | |
| NB175 | M84R/ | 0.04/0.08 | 0.09/0.04 | <0.01/- | DNI | 0.2/0.04 | 18/0.2 | 0.5/- | -/- |
| | V81R/ | | | | | | | | |
| | K112M | | | | | | | | |
| NB141 | M84R/ | 18.7/1.2 | 0.07/0.2 | DNI/- | 1.6/11.6 | 0.2/0.2 | DNI/0.6 | DNI/- | 0.2/- |
| | S82H | | | | | | | | |
| NB145 | M84R/ | 0.01/0.02 | 0.1/0.09 | DNI/- | >1/0.7 | 0.2/0.02 | DNI/0.26 | 2.3/- | 0.5/- |
| | K112M | | | | | | | | |
| NB178 | M84R/ | 0.03/0.07 | 0.07/0.3 | 0.004/- | 3.1/3.5 | 0.04/0.09 | 6.9/0.5 | 0.2/- | -/- |
| | V81G/ | | | | | | | | |
| | K112M | | | | | | | | |

*Ki against the human and mice proteases are shown as “human/mice”.
DNI: do not inhibit.
“-”: data unavailable.

TABLE 3
Amino Acid Sequences of Preliminary and
Optimized Lead Candidates with Leader Sequence

| SEQ ID | Peptide | Mutation | Amino Acid Sequence |
|---|---|---|---|
| 1 | NB101 | wt | MKTILPAVLFAAFATTSAWAA |
| | | | ESVQPLEKIAPYPQAEKGMKR |
| | | | QVIQLTPQEDESTLKVELLIG |
| | | | QTLEVDCNLHRLGGKLENKTL |
| | | | EGWGYDYYVFDKVSSPVSTMM |
| | | | ACPDGKKEKKFVTAYLGDAGM |
| | | | LRYNSKLPIVVYTPDNVDVKY |
| | | | RVWKAEEKIDNAVVR |
| 2 | NB109 | M84R | MKTILPAVLFAAFATTSAWAA |
| | | | ESVQPLEKIAPYPQAEKGMKR |
| | | | QVIQLTPQEDESTLKVELLIG |
| | | | QTLEVDCNLHRLGGKLENKTL |
| | | | EGWGYDYYVFDKVSSPVSTRM |
| | | | ACPDGKKEKKFVTAYLGDAGM |
| | | | LRYNSKLPIVVYTPDNVDVKY |
| | | | RVWKAEEKIDNAVVR |
| 3 | NB142 | M84R/ | MKTILPAVLFAAFATTSAWAA |
| | | V81R | ESVQPLEKIAPYPQAEKGMKR |
| | | | QVIQLTPQEDESTLKVELLIG |
| | | | QTLEVDCNLHRLGGKLENKTL |
| | | | EGWGYDYYVFDKVSSPRSTRM |
| | | | ACPDGKKEKKFVTAYLGDAGM |
| | | | LRYNSKLPIVVYTPDNVDVKY |
| | | | RVWKAEEKIDNAVVR |
| 4 | NB137 | M84R/ | MKTILPAVLFAAFATTSAWAA |
| | | K76I | ESVQPLEKIAPYPQAEKGMKR |
| | | | QVIQLTPQEDESTLKVELLIG |
| | | | QTLEVDCNLHRLGGKLENKTL |
| | | | EGWGYDYYVFDIVSSPVSTRM |
| | | | ACPDGKKEKKFVTAYLGDAGM |
| | | | LRYNSKLPIVVYTPDNVDVKY |
| | | | RVWKAEEKIDNAVVR |
| 5 | NB147 | M84R/ | MKTILPAVLFAAFATTSAWAA |
| | | R54D | ESVQPLEKIAPYPQAEKGMKR |
| | | | QVIQLTPQEDESTLKVELLIG |
| | | | QTLEVDCNLHDLGGKLENKTL |
| | | | EGWGYDYYVFDKVSSPVSTRM |
| | | | ACPDGKKEKKFVTAYLGDAGM |
| | | | LRYNSKLPIVVYTPDNVDVKY |
| | | | RVWKAEEKIDNAVVR |
| 6 | NB175 | M84R/ | MKTILPAVLFAAFATTSAWAA |
| | | V81R/ | ESVQPLEKIAPYPQAEKGMKR |
| | | K112M | QVIQLTPQEDESTLKVELLIG |
| | | | QTLEVDCNLHRLGGKLENKTL |
| | | | EGWGYDYYVFDKVSSPRSTRM |
| | | | ACPDGKKEKKFVTAYLGDAGM |
| | | | LRYNSMLPIVVYTPDNVDVKY |
| | | | RVWKAEEKIDNAVVR |
| 7 | NB141 | M84R/ | MKTILPAVLFAAFATTSAWAA |
| | | S821I | ESVQPLEKIAPYPQAEKGMKR |
| | | | QVIQLTPQEDESTLKVELLIG |
| | | | QTLEVDCNLHRLGGKLENKTL |
| | | | EGWGYDYYVFDKVSSPVHTRM |
| | | | ACPDGKKEKKFVTAYLGDAGM |
| | | | LRYNSKLPIVVYTPDNVDVKY |
| | | | RVWKAEEKIDNAVVR |
| 8 | NB145 | M84R/ | MKTILPAVLFAAFATTSAWAA |
| | | K112M | ESVQPLEKIAPYPQAEKGMKR |
| | | | QVIQLTPQEDESTLKVELLIG |
| | | | QTLEVDCNLHRLGGKLENKTL |
| | | | EGWGYDYYVFDKVSSPVSTRM |
| | | | ACPDGKKEKKFVTAYLGDAGM |
| | | | LRYNSMLPIVVYTPDNVDVKY |
| | | | RVWKAEEKIDNAVVR |
| 9 | NB178 | M84R/ | MKTILPAVLFAAFATTSAWAA |
| | | V81G/ | ESVQPLEKIAPYPQAEKGMKR |
| | | K112M | QVIQLTPQEDESTLKVELLIG |
| | | | QTLEVDCNLHRLGGKLENKTL |
| | | | EGWGYDYYVFDKVSSPGSTRM |
| | | | ACPDGKKEKKFVTAYLGDAGM |
| | | | LRYNSMLPIVVYTPDNVDVKY |
| | | | RVWKAEEKIDNAVVR |

TABLE 4
Amino Acid Sequences of Preliminary and
Optimized Lead Candidates without Leader Sequence

| SEQ ID | Peptide | Mutation | Amino Acid Sequence |
|---|---|---|---|
| 10 | NB101 | wt | AESVQPLEKIAPYPQAEKGMK |
| | | | RQVIQLTPQEDESTLKVELLI |
| | | | GQTLEVDCNLHRLGGKLENKT |
| | | | LEGWGYDYYVFDKVSSPVSTM |
| | | | MACPDGKKEKKFVTAYLGDAG |
| | | | MLRYNSKLPIVVYTPDNVDVK |
| | | | YRVWKAEEKIDNAVVR |
| 11 | NB109 | M84R | AESVQPLEKIAPYPQAEKGMK |
| | | | RQVIQLTPQEDESTLKVELLI |
| | | | GQTLEVDCNLHRLGGKLENKT |
| | | | LEGWGYDYYVFDKVSSPVSTR |
| | | | MACPDGKKEKKFVTAYLGDAG |
| | | | MLRYNSKLPIVVYTPDNVDVK |
| | | | YRVWKAEEKIDNAVVR |
| 12 | NB142 | M84R/ | AESVQPLEKIAPYPQAEKGMK |
| | | V81R | RQVIQLTPQEDESTLKVELLI |
| | | | GQTLEVDCNLHRLGGKLENKT |
| | | | LEGWGYDYYVFDKVSSPRSTR |
| | | | MACPDGKKEKKFVTAYLGDAG |
| | | | MLRYNSKLPIVVYTPDNVDVK |
| | | | YRVWKAEEKIDNAVVR |
| 13 | NB137 | M84R/ | AESVQPLEKIAPYPQAEKGMK |
| | | K76I | RQVIQLTPQEDESTLKVELLI |
| | | | GQTLEVDCNLHRLGGKLENKT |
| | | | LEGWGYDYYVFDIVSSPVSTR |
| | | | MACPDGKKEKKFVTAYLGDAG |
| | | | MLRYNSKLPIVVYTPDNVDVK |
| | | | YRVWKAEEKIDNAVVR |
| 14 | NB147 | M84R/ | AESVQPLEKIAPYPQAEKGMK |
| | | R54D | RQVIQLTPQEDESTLKVELLI |
| | | | GQTLEVDCNLHDLGGKLENKT |
| | | | LEGWGYDYYVFDKVSSPVSTR |
| | | | MACPDGKKEKKFVTAYLGDAG |
| | | | MLRYNSKLPIVVYTPDNVDVK |
| | | | YRVWKAEEKIDNAVVR |
| 15 | NB175 | M84R/ | AESVQPLEKIAPYPQAEKGMK |
| | | V81R/ | RQVIQLTPQEDESTLKVELLI |
| | | K112M | GQTLEVDCNLHRLGGKLENKT |
| | | | LEGWGYDYYVFDKVSSPRSTR |
| | | | MACPDGKKEKKFVTAYLGDAG |
| | | | MLRYNSMLPIVVYTPDNVDVK |
| | | | YRVWKAEEKIDNAVVR |
| 16 | NB141 | M84R/ | AESVQPLEKIAPYPQAEKGMK |
| | | S821I | RQVIQLTPQEDESTLKVELLI |
| | | | GQTLEVDCNLHRLGGKLENKT |
| | | | LEGWGYDYYVFDKVSSPVHTR |
| | | | MACPDGKKEKKFVTAYLGDAG |
| | | | MLRYNSKLPIVVYTPDNVDVK |
| | | | YRVWKAEEKIDNAVVR |
| 17 | NB145 | M84R/ | AESVQPLEKIAPYPQAEKGMK |
| | | K112M | RQVIQLTPQEDESTLKVELLI |
| | | | GQTLEVDCNLHRLGGKLENKT |
| | | | LEGWGYDYYVFDKVSSPVSTR |
| | | | MACPDGKKEKKFVTAYLGDAG |
| | | | MLRYNSMLPIVVYTPDNVDVK |
| | | | YRVWKAEEKIDNAVVR |
| 18 | NB178 | M84R/ | AESVQPLEKIAPYPQAEKGMK |
| | | V81G/ | RQVIQLTPQEDESTLKVELLI |
| | | K112M | GQTLEVDCNLHRLGGKLENKT |
| | | | LEGWGYDYYVFDKVSSPGSTR |
| | | | MACPDGKKEKKFVTAYLGDAG |
| | | | MLRYNSMLPIVVYTPDNVDVK |
| | | | YRVWKAEEKIDNAVVR |

Efficacy in Endotoxemia Model

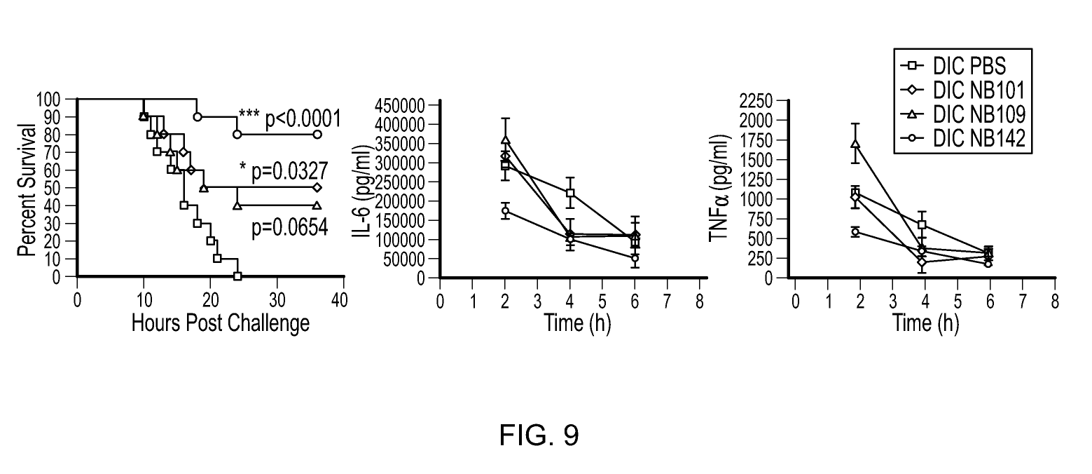

Murine endotoxemia model was used as the first-line screening model due to its simplicity. All of the potentially optimized lead candidates protected animals in this model; NB142, NB137, NB147, and NB178 appeared to be the most effective ones. Interestingly, NB142 is significantly superior to NB101 or NB109 in this model (Error! Reference source not found.9). In addition to having the highest rate of animal survival, NB142 also was most effective at inhibiting inflammatory cytokines IL-6 and TNF-α (FIG. 9)

Preliminary Efficacy in VHF Models

Several of the peptides shown in Table 3 in mice challenged with injections of poly(I:C), an inosine polymer resembling foreign RNA molecules. Since VHF viruses are all RNA viruses, this model is designed to replicate host responses to viral RNA molecules. Similar to VHF viruses, poly(I:C) injection triggers signs of SIRS, including release of inflammatory cytokines, elevated D-dimers (a product of fibrinolysis indicative of DIC), and abundant micro-thrombi in the liver, lung, and kidneys.

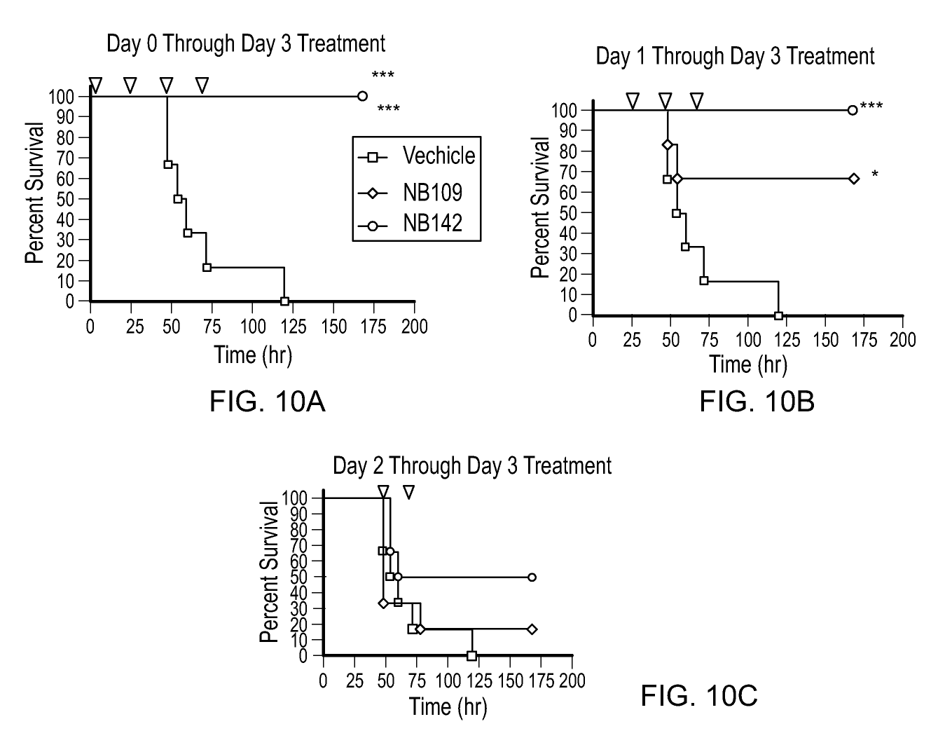

Untreated animals invariably died in five days after the first poly(I:C) injection. When treatment was initiated prior to poly(I:C) injection, NB109, NB142, NB137, and NB147 all significantly prevented animal death. When NB109 treatment was initiated after poly(I:C) injection, it was effective when it was first given at one day after challenge (FIG. 1). NB104 prolonged animal survival even when initiated at 48 hrs after poly(I:C) challenge with only two treatments (FIG. 10). This result suggests that both NB142 and NB109 can prevent SIRS at the time of induction, but NB142 may be more effective at treating established SIRS associated with VHF.

In the same model, when given prior to poly(I:C) challenge, NB101, NB109, and NB142 all significantly inhibited inflammatory cytokines and D-dimer triggered by poly(I:C). However, among the three candidates, NB142 was the most effective at inhibiting inflammatory cytokines IL-6 and TNF-α (Error! Reference source not found.).

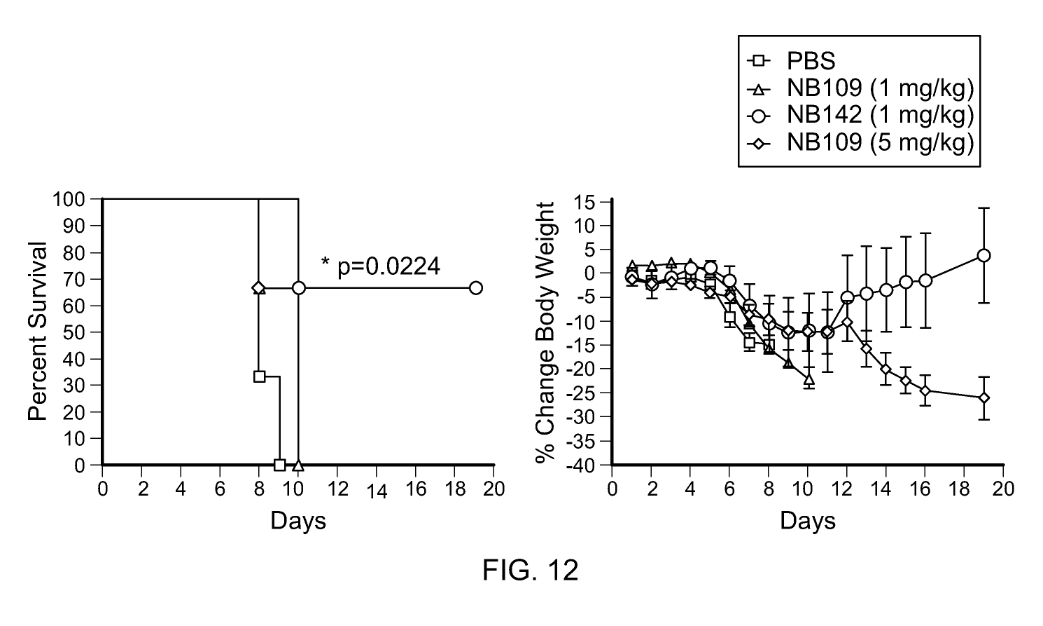

Next, NB109 and NB142 were compared in a study of guinea pigs infected with Zaire strain of EBOV. While vehicle-treated animals invariably died by Day 9, NB142 at 1 mg/kg/day and NB109 at 5 mg/kg/day achieved significant, 67% survival. Again, NB142 showed superior efficacy, with better survival at a lower dose and remarkable body weight gains (FIG. 12). The strength of this study result also lies in the fact that NB109 and NB142 treatment was with an unoptimized treatment dose and regime initiated at 24 hr post infection.

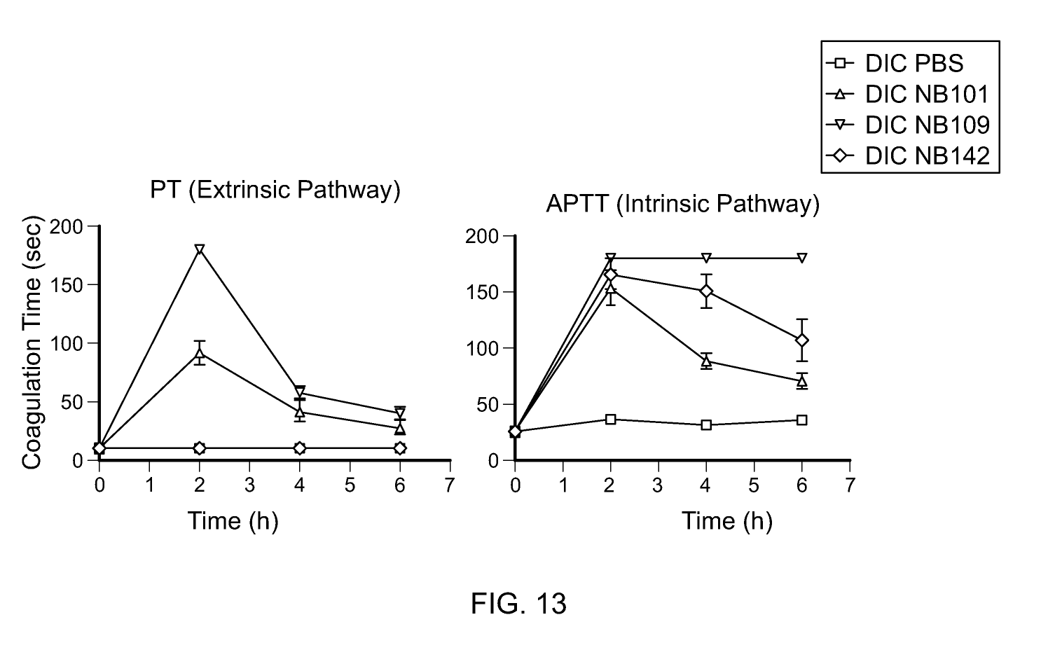

Preliminary Pharmacodynamics of NB142, NB101 and NB109 NB142 has distinct pharmacodynamics (PD) from NB101 and NB109 in vivo. While NB101 and NB109 both cause PT elevations, NB142 does not affect PT (FIG. 13). All three candidates cause elevation in aPTT with various potencies. The PD result indicates that NB101 and NB109 inhibit both extrinsic and intrinsic coagulation pathways, whereas NB142 appears to specifically affect the intrinsic coagulation pathway.

Hematological monitoring of EBOV infected rhesus monkeys reveals that consumptive coagulopathy in EBOV HF is due to activation of the intrinsic coagulative pathway, rather than extrinsic coagulative pathway [66]. Intrinsic coagulative pathway is directly activated by inflammatory cytokines and kallikrein, and is potentiated by plasmin activation. NB142 has anti-inflammatory effects. It also potently inhibits kallikrein and plasmin while sparing thrombin. Thus it may inhibit the upstream events that trigger intrinsic coagulation without exacerbating consumptive coagulopathy. Therefore, NB142 may have a preferred PD profile for VHF treatment.

Drug Substance

Peptide can be produced using a high-density, fed-batch E. coli fermentation process followed by periplasmic extraction, an ion-exchange chromatography, and a filtration step to remove endotoxin.

Fermentation Process

Two microbial expression systems can be evaluated for lead compound production: E. coli and yeast. NB109 is produced using a time dependent fed-batch E. coli fermentation process using glucose as the carbon source that yields ˜0.2 gm purified NB109 per liter of fermentation. The lead compounds can also be produced with a dissolved oxygen-dependent feed control system that uses glycerol as a carbon source. This fermentation process has resulted >9 grams per liter expression of a different protein drug candidate. This latter process can be easily scaled up. It uses a semi-defined medium composed of USP-grade reagents that are certified animal-free.

As an alternative to the bacterial expression system, yeast strains such as P. pastoris and H. polymorpha can also be evaluated as a system for production lead compounds. These have the advantages of higher eukaryotic expression systems such as better protein processing, folding and secretion when compared to microbial systems, and still have rapid growth and tightly regulated promoters. Peptides can be expressed by secretion into yeast media to greatly simplify the purification process. As part of the present invention, strains of P. pastoris have been generated to secrete lead compounds into yeast media. These strains are methanol-inducible and amenable to fermentation.

Further optimization of the P. pastoris system is possible by investigating multiple secretion leader sequences such as a-mating factor, a-amylase, glucoamylase, inulinase, and invertase yeast signal sequences, and transforming multiple wild type and protease deficient yeast strains. Screening of colonies can be performed from supernatants of small scale cultures grown in 96- and 24-well formats. Selected clones can be grown in shaker flask culture before transfer to fermentation. The fermentation process can be established using available BioFlo 3000 and BioFlo IV fermenters with volumes of 4 to 20 liters. Methanol feed for induction of expression can be quantified by an available YSI 2700 Select Biochemistry Analyzer with methanol probe. Fermentation optimization can vary media and feed composition, pH, temperature, feed time course, and time of induction to achieve desired levels of protein expression.

Purification Process

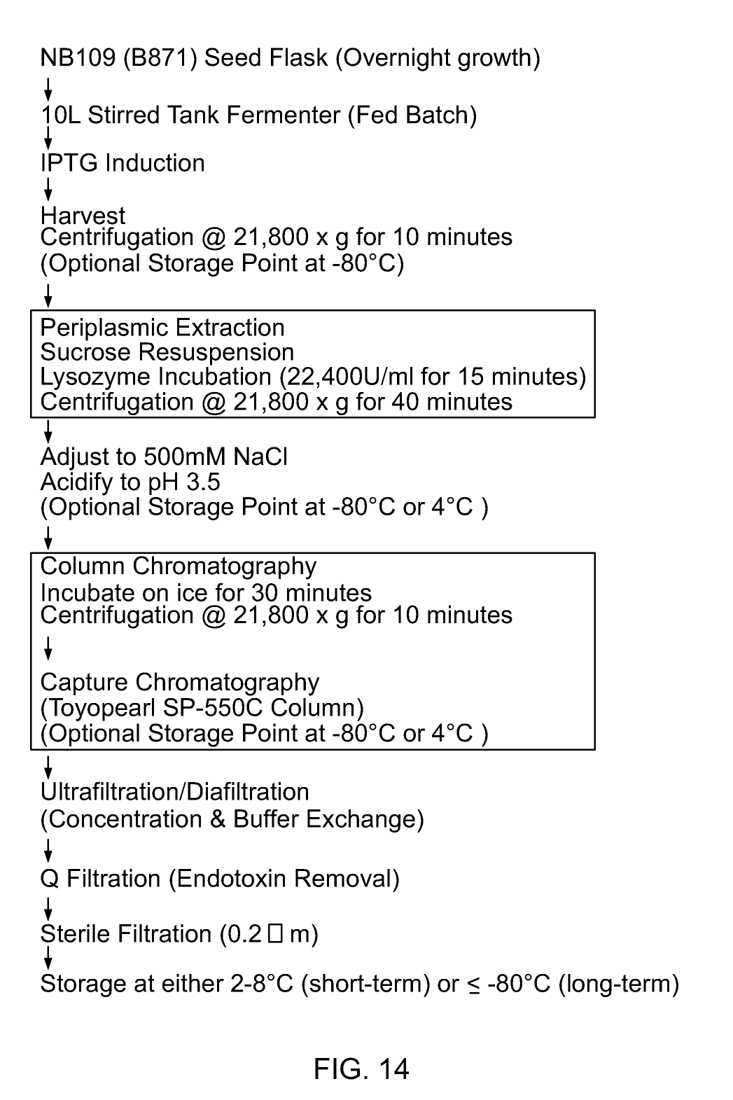

The purification process from E. coli fermentation involves a periplasmic extraction followed by an ion-exchange chromatography step for purification and an ion-exchange filtration step for endotoxin reduction. This purification has worked for peptides described herein. The details of this process are presented in FIG. 14.

Additional downstream steps can include, but are not limited to, affinity chromatography, hydrophobic interaction chromatography, size-exclusion chromatography, and additional ion-exchange steps. Initial screening can be performed in 96-well filter plates for high throughput without using robotics. Binding conditions to be evaluated can include chromatography resins, salts, ionic strength, and pH. Micro-eluates can be analyzed for overall concentration by UV absorbance using an available 96-well UV spectrophotometer and purity by 48-sample SDS-PAGE (Invitrogen, Carlsbad, Calif.) with Coomassie staining. Select conditions can be scaled up to chromatography using standard 1-10 ml columns on available FPLCs. Yield and purity of the process intermediates can be monitored using a subset of the release tests described below, including SDS-PAGE, HPLC and activity.

Development can also focus on adapting the purification process to the yeast expression system and adding additional purification steps to enhance purity. Additional steps may include, but are not limited to, hydrophobic interaction chromatography, reversed-phase chromatography, and additional ion-exchange steps.

Pre-Formulation and Formulation Development

The lead compounds can be developed into a sterile, non-preserved, unit-dose parenteral product. Current data indicate that the lead compounds can be very robust and stable over a broad range of pH and temperature.

Estimated Dosage

Based on the 1 mg/kg/day effective dose of NB142 in the guinea pig EBOV model, the human treatment dose could be approximately 0.2 mg/kg/day. For a maximum of 7-day course, the estimated total drug consumption would be 84 mg (for 60 kg individual) to 280 mg (for 200 kg individual).

A number of embodiments of the invention have been described. Nevertheless, it will be understood that various modifications may be made without departing from the spirit and scope of the invention.

### REFERENCE LIST

1. Borio L, Inglesby T, Peters C J, Schmaljohn A L, Hughes J M, Jahrling P B, Ksiazek T, Johnson K M, Meyerhoff A, O'Toole T, Ascher M S, Bartlett J, Breman J G, Eitzen E M, Jr., Hamburg M, Hauer J, Henderson D A, Johnson R T, Kwik G, Layton M, Lillibridge S, Nabel G J, Osterholm M T, Perl T M, Russell P, Tonat K (2002) Hemorrhagic fever viruses as biological weapons: medical and public health management. JAMA 287:2391-2405
2. Mahanty S, Bray M (2004) Pathogenesis of filoviral haemorrhagic fevers. Lancet Infect Dis 4:487-498
3. Hoenen T, Groseth A, Falzarano D, Feldmann H (2006) Ebola virus: unravelling pathogenesis to combat a deadly disease. Trends Mol Med 12:206-215
4. Chen J P, Cosgriff T M (2000) Hemorrhagic fever virus-induced changes in hemostasis and vascular biology. Blood Coagulation and Fibrinolysis 11:461-483
5. Borio L, Inglesby T, Peters C J, Schmaljohn A L, Hughes J M, Jahrling P B, Ksiazek T, Johnson K M, Meyerhoff A, O'Toole T, Ascher M S, Bartlett J, Breman J G, Eitzen E M, Jr., Hamburg M, Hauer J, Henderson D A, Johnson R T, Kwik G, Layton M, Lillibridge S, Nabel G J, Osterholm M T, Perl T M, Russell P, Tonat K (2002) Hemorrhagic fever viruses as biological weapons: medical and public health management. JAMA 287:2391-2405
6. Hensley L E, Stevens E L, Yan S B, Geisbert J B, Macias W L, Larsen T, ddario-DiCaprio K M, Cassell G H, Jahrling P B, Geisbert T W (2007) Recombinant human activated protein C for the postexposure treatment of Ebola hemorrhagic fever. J Infect Dis 196 Suppl 2:S390-S399
7. Stutz A, Golenbock D T, Latz E (2009) Inflammasomes: too big to miss. J Clin Invest 119:3502-3511
8. Bhatia M, He M, Zhang H, Moochhala S (2009) Sepsis as a model of SIRS. Front Biosci 14:4703-4711
9. Roumen R M, Hendriks T, van d, V, Nieuwenhuijzen G A, Sauerwein R W, van der Meer J W, Goris R J (1993) Cytokine patterns in patients after major vascular surgery, hemorrhagic shock, and severe blunt trauma. Relation with subsequent adult respiratory distress syndrome and multiple organ failure. Ann Surg 218:769-776
10. Kumar A T, Sudhir U, Punith K, Kumar R, Ravi K, V, Rao M Y (2009) Cytokine profile in elderly patients with sepsis. Indian J Crit Care Med 13:74-78
11. Mavrommatis A C, Theodoridis T, Economou M, Kotanidou A, El A M, Christopoulou-Kokkinou V, Zakynthinos S G (2001) Activation of the fibrinolytic system and utilization of the coagulation inhibitors in sepsis: comparison with severe sepsis and septic shock. Intensive Care Med 27:1853-1859
12. Seligsohn U (2007) Factor XI in haemostasis and thrombosis: past, present and future. Thromb Haemost 98:84-89
13. Hack C E (2000) Tissue factor pathway of coagulation in sepsis. Crit Care Med 28:S25-S30
14. Lolis E, Bucala R (2003) Therapeutic approaches to innate immunity: severe sepsis and septic shock. Nat Rev Drug Discov 2:635-645
15. Satran R, Almog Y (2003) The coagulopathy of sepsis: pathophysiology and management. Isr Med Assoc J 5:516-520
16. Dellinger R P (2003) Inflammation and coagulation: implications for the septic patient. Clin Infect Dis 36:1259-1265
17. Schouten M, Wiersinga W J, Levi M, van der P T (2008) Inflammation, endothelium, and coagulation in sepsis. J Leukoc Biol 83:536-545
18. Jean-Baptiste E (2007) Cellular mechanisms in sepsis. J Intensive Care Med 22:63-72
19. Jansen P M, Pixley R A, Brouwer M, de J, I, Chang A C, Hack C E, Taylor F B, Jr., Colman R W (1996) Inhibition of factor XII in septic baboons attenuates the activation of complement and fibrinolytic systems and reduces the release of interleukin-6 and neutrophil elastase. Blood 87:2337-2344
20. Zeerleder S, Caliezi C, van M G, Eerenberg-Belmer A, Sulzer I, Hack C E, Wuillemin W A (2003) Administration of C1 inhibitor reduces neutrophil activation in patients with sepsis. Clin Diagn Lab Immunol 10:529-535
21. Hack C E, Colman R W (1999) The Role of the Contact System in the Pathogenesis of Septic Shock. Sepsis 3:111-1
22. Schmid A, Eich-Rathfelder S, Whalley E T, Cheronis J C, Scheuber H P, Fritz H, Siebeck M (1998) Endogenous B1 receptor mediated hypotension produced by contact system activation in the presence of endotoxemia. Immunopharmacology 40:131-137
23. Pixley R A, De La C R, Page J D, Kaufman N, Wyshock E G, Chang A, Taylor F B, Jr., Colman R W (1993) The contact system contributes to hypotension but not disseminated intravascular coagulation in lethal bacteremia. In vivo use of a monoclonal anti-factor XII antibody to block contact activation in baboons. J Clin Invest 91:61-68
24. Schmidt C, Hocherl K, Kurt B, Moritz S, Kurtz A, Bucher M (2009) Blockade of multiple but not single cytokines abrogates downregulation of angiotensin II type-I receptors and anticipates septic shock. Cytokine
25. Witte J, Jochum M, Scherer R, Schramm W, Hochstrasser K, Fritz H (1982) Disturbances of selected plasma proteins in hyperdynamic septic shock. Intensive Care Med 8:215-222
26. Ergonul O (2009) DEBATE (see Elaldi N et al, Efficacy of oral ribavirin treatment in Crimean-Congo haemorrhagic fever: a quasi-experimental study from Turkey. Journal of Infection 2009; 58: 238-244): Biases and misinterpretation in the assessment of the efficacy of oral ribavirin in the treatment of Crimean-Congo hemorrhagic fever. J Infect 59:284-286
27. Gowen B B, Smee D F, Wong M H, Hall J O, Jung K H, Bailey K W, Stevens J R, Furuta Y, Morrey J D (2008) Treatment of late stage disease in a model of arenaviral hemorrhagic fever: T-705 efficacy and reduced toxicity suggests an alternative to ribavirin. PLoS One 3:e3725
28. Gowen B B, Wong M H, Jung K H, Sanders A B, Mendenhall M, Bailey K W, Furuta Y, Sidwell R W (2007) In vitro and in vivo activities of T-705 against arenavirus and bunyavirus infections. Antimicrob Agents Chemother 51:3168-3176
29. Opal S M, Patrozou E (2009) Translational research in the development of novel sepsis therapeutics: logical deductive reasoning or mission impossible? Crit Care Med 37:S10-S15
30. Cross A S, Opal S M (2003) A new paradigm for the treatment of sepsis: is it time to consider combination therapy? Ann Intern Med 138:502-505
31. Schmidt C, Hocherl K, Kurt B, Moritz S, Kurtz A, Bucher M (2009) Blockade of multiple but not single cytokines abrogates downregulation of angiotensin II type-I receptors and anticipates septic shock. Cytokine
32. Minneci P C, Deans K J, Cui X, Banks S M, Natanson C, Eichacker P Q (2006) Antithrombotic therapies for sepsis: a need for more studies. Crit Care Med 34:538-541
33. Riedemann N C, Guo R F, Ward P A (2003) The enigma of sepsis. J Clin Invest 112:460-467
34. Deans K J, Haley M, Natanson C, Eichacker P Q, Minneci P C (2005) Novel therapies for sepsis: a review. J Trauma 58:867-874
35. LaRosa S P, Opal S M (2005) Tissue factor pathway inhibitor and antithrombin trial results. Crit Care Clin 21:433-448
36. Abraham E, Laterre P F, Garg R, Levy H, Talwar D, Trzaskoma B L, Francois B, Guy J S, Bruckmann M, Rea-Neto A, Rossaint R, Perrotin D, Sablotzki A, Arkins N, Utterback B G, Macias W L (2005) Drotrecogin alfa (activated) for adults with severe sepsis and a low risk of death. N Engl J Med 353:1332-1341
37. Eichacker P Q, Natanson C (2007) Increasing evidence that the risks of rhAPC may outweigh its benefits. Intensive Care Med 33:396-399
38. Geisbert T W, Hensley L E, Jahrling P B, Larsen T, Geisbert J B, Paragas J, Young H A, Fredeking T M, Rote W E, Vlasuk G P (2003) Treatment of Ebola virus infection with a recombinant inhibitor of factor VIIa/tissue factor: a study in rhesus monkeys. Lancet 362:1953-1958
39. Geisbert T W, ddario-DiCaprio K M, Geisbert J B, Young H A, Formenty P, Fritz E A, Larsen T, Hensley L E (2007) Marburg virus Angola infection of rhesus macaques: pathogenesis and treatment with recombinant nematode anticoagulant protein c2. J Infect Dis 196 Suppl 2:S372-S381
40. Weijer S, Schoenmakers S H, Florquin S, Levi M, Vlasuk G P, Rote W E, Reitsma P H, Spek C A, van der P T (2004) Inhibition of the tissue factor/factor VIIa pathway does not influence the inflammatory or antibacterial response to abdominal sepsis induced by Escherichia coli in mice. J Infect Dis 189:2308-2317
41. Moons A H, Peters R J, Cate H, Bauer K A, Vlasuk G P, Buller H R, Levi M (2002) Recombinant nematode anticoagulant protein c2, a novel inhibitor of tissue factor-factor VIIa activity, abrogates endotoxin-induced coagulation in chimpanzees. Thromb Haemost 88:627-631
42. Taylor F B, Jr., Wada H, Kinasewitz G (2000) Description of compensated and uncompensated disseminated intravascular coagulation (DIC) responses (non-overt and overt DIC) in baboon models of intravenous and intraperitoneal Escherichia coli sepsis and in the human model of endotoxemia: toward a better definition of DIC. Crit Care Med 28:S12-S19
43. Juffrie M, van Der Meer G M, Hack C E, Haasnoot K, Sutaryo, Veerman A J, Thijs L G (2000) Inflammatory mediators in dengue virus infection in children: interleukin-8 and its relationship to neutrophil degranulation. Infect Immun 68:702-707
44. Pacher R, Redl H, Frass M, Petzl D H, Schuster E, Woloszczuk W (1989) Relationship between neopterin and granulocyte elastase plasma levels and the severity of multiple organ failure. Crit Care Med 17:221-226
45. Samis J A, Stewart K A, Nesheim M E, Taylor F B, Jr. (2007) Factor V cleavage and inactivation are temporally associated with elevated elastase during experimental sepsis. J Thromb Haemost 5:2559-2561
46. Matsumoto T, Wada H, Nobori T, Nakatani K, Onishi K, Nishikawa M, Shiku H, Kazahaya Y, Sawai T, Koike K, Matsuda M (2005) Elevated plasma levels of fibrin degradation products by granulocyte-derived elastase in patients with disseminated intravascular coagulation. Clin Appl Thromb Hemost 11:391-400
47. Tralau T, Meyer-Hoffert U, Schroder J M, Wiedow O (2004) Human leukocyte elastase and cathepsin G are specific inhibitors of C5a-dependent neutrophil enzyme release and chemotaxis. Exp Dermatol 13:316-325
48. Siebeck M, Hoffmann H, Weipert J, Fritz H (1992) Effect of the elastase inhibitor eglin C in porcine endotoxin shock. Circ Shock 36:174-179
49. Tamakuma S, Ogawa M, Aikawa N, Kubota T, Hirasawa H, Ishizaka A, Taenaka N, Hamada C, Matsuoka S, Abiru T (2004) Relationship between neutrophil elastase and acute lung injury in humans. Pulm Pharmacol Ther 17:271-279
50. Zeiher B G, Artigas A, Vincent J L, Dmitrienko A, Jackson K, Thompson B T, Bernard G (2004) Neutrophil elastase inhibition in acute lung injury: results of the STRIVE study. Crit Care Med 32:1695-1702
51. Sallenave J M (2000) The role of secretory leukocyte proteinase inhibitor and elafin (elastase-specific inhibitor/skin-derived antileukoprotease) as alarm antiproteinases in inflammatory lung disease. Respir Res 1:87-92
52. Zani M L, Baranger K, Guyot N, let-Choisy S, Moreau T (2009) Protease inhibitors derived from elafin and SLPI and engineered to have enhanced specificity towards neutrophil serine proteases. Protein Sci 18:579-594
53. Grobmyer S R, Barie P S, Nathan C F, Fuortes M, Lin E, Lowry S F, Wright C D, Weyant MJ , Hydo L, Reeves F, Shiloh M U, Ding A (2000) Secretory leukocyte protease inhibitor, an inhibitor of neutrophil activation, is elevated in serum in human sepsis and experimental endotoxemia. Crit Care Med 28:1276-1282
54. Abildgaard U (2007) Antithrombin—early prophecies and present challenges. Thromb Haemost 98:97-104
55. Abildgaard U (2007) Antithrombin—early prophecies and present challenges. Thromb Haemost 98:97-104
56. Hileman R E, Fromm J R, Weiler J M, Linhardt R J (1998) Glycosaminoglycan-protein interactions: definition of consensus sites in glycosaminoglycan binding proteins. Bioessays 20:156-167
57. Abildgaard U (2007) Antithrombin—early prophecies and present challenges. Thromb Haemost 98:97-104
58. Abildgaard U (2007) Antithrombin—early prophecies and present challenges.

Thromb Haemost 98:97-104
59. Gettins P G (2002) Serpin structure, mechanism, and function. Chem Rev 102:4751-4804
60. Horn J K (2003) Bacterial agents used for bioterrorism. Surg Infect (Larchmt) 4:281-287
61. Gasteiger E, Gattiker A, Hoogland C, Ivanyi I, Appel R D, Bairoch A (2003) ExPASy: The proteomics server for in-depth protein knowledge and analysis. Nucleic Acids Res 31:3784-3788
62. Colman R W, Clowes A W, George J N, Goldhaber S Z, Marder V J (2006) Overview of Hemostasis. In: Colman R W, Marder V J, Clowes A W, George J N, Goldhaber S Z (eds) Hemostasis and Thrombosis Basic Principles and Clinical Practice. Lippincott Williams & Wilkins, Philadelphia, pp 3-16
63. Brozna J P (1990) Shwartzman reaction. Semin Thromb Hemost 16:326-332
64. Slofstra S H, van', V, Buurman W A, Reitsma P H, ten C H, Spek C A (2005) Low molecular weight heparin attenuates multiple organ failure in a murine model of disseminated intravascular coagulation. Crit Care Med 33:1365-1370
65. Hubbard W J, Choudhry M, Schwacha M G, Kerby J D, Rue L W, III, Bland K I, Chaudry I H (2005) Cecal ligation and puncture. Shock 24 Suppl 1:52-57
66. Fisher-Hoch S P, Platt G S, LLoyd G, Simpson D I H (1983) Haematological and biochemical monitoring of Ebola infection in rhesus monkeys: implications for patient management. The Lancet1055-1058
67. Sidwell R W, Smee D F (2003) Viruses of the Bunya- and Togaviridae families: potential as bioterrorism agents and means of control. Antiviral Res 57:101-111
68. Nooteboom A, van der Linden C J, Hendriks T (2004) Modulation of adhesion molecule expression on endothelial cells after induction by lipopolysaccharide-stimulated whole blood. Scand J Immunol 59:440-448
69. Nooteboom A, van der Linden C J, Hendriks T (2004) Modulation of adhesion molecule expression on endothelial cells after induction by lipopolysaccharide-stimulated whole blood. Scand J Immunol 59:440-448
70. Schildberger A, Rossmanith E, Weber V, Falkenhagen D (2009) Monitoring of endothelial cell activation in experimental sepsis with a two-step cell culture model. Innate Immun

1. A polypeptide comprising the amino acid sequence of any of SEQ ID NOs: 2-9 and 11-18.

2. A polypeptide comprising the amino acid sequence of any of SEQ ID NO:11-18 preceded by a methionine.

3. A pharmaceutical composition comprising a polypeptide of claim 1 and a pharmaceutically acceptable carrier or excipient.

4. A method for treating a patient infected with a microorganism that causes viral hemorrhagic fever, the method comprising administering the pharmaceutical composition of claim 3.

5. The method of claim 4 wherein the patient is infected with a virus from a family selected from the group consisting of: Filoviridae, Bunyaviridae, Flaviviridae, and Arenaviridae.

6. The method of claim 5 wherein the patient is infected with a virus selected from Ebola virus (EBOV), Marburg Virus (MARV), Rift Valley Fever virus (RVFV), Hantaviruses, and Dengue virus.

7. A polypeptide comprising the amino acid sequence of any of SEQ ID NO:2-9 and 11-18 with up to 5 single amino acid changes or deletions provided that the polypeptide does not comprise the amino acid sequence of SEQ ID NO:1 or 10

8. The polypeptide of claim 7 having up to 3 single amino acid changes provided that the polypeptide does not comprise the amino acid sequence of SEQ ID NO:1 or 10

9. The polypeptide of claim 7 having up to 3 single amino acid changes provided that the polypeptide does not comprise the amino acid sequence of SEQ ID NO:1 or 10

10. The polypeptide of claim 7 having up to 2 single amino acid changes provided that the polypeptide does not comprise the amino acid sequence of SEQ ID NO:1 or 10

11. The polypeptide of claim 7 having no more that one amino acid change provided that the polypeptide does not comprise the amino acid sequence of SEQ ID NO:1 or 10

12. A polypeptide comprising the amino acid sequence of any of SEQ ID NO:11-18 with up to 5 single amino acid changes or deletions provided that the polypeptide does not comprise the amino acid sequence of SEQ ID NO:10

13. The polypeptide of claim 12 having up to 3 single amino acid changes provided that the polypeptide does not comprise the amino acid sequence of SEQ ID NO:10

14. The polypeptide of claim 12 having up to 3 single amino acid changes provided that the polypeptide does not comprise the amino acid sequence of SEQ ID NO:10.

15. The polypeptide of claim 12 having up to 2 single amino acid changes provided that the polypeptide does not comprise the amino acid sequence of SEQ ID NO:10

16. The polypeptide of claim 12 having no more that one amino acid change provided that the polypeptide does not comprise the amino acid sequence of SEQ ID NO:10.

17. (canceled)

18. A pharmaceutical composition comprising a polypeptide of claim 7 and a pharmaceutically acceptable carrier or excipient.

19. A method for treating a patient infected with a microorganism that causes viral hemorrhagic fever, the method comprising administering the pharmaceutical composition of claim 18.

20. The method of claim 19 wherein the patient is infected with a virus from a family selected from the group consisting of: Filoviridae, Bunyaviridae, Flaviviridae, and Arenaviridae.

21. The method of claim 20 wherein the patient is infected with a virus selected from Ebola virus (EBOV), Marburg Virus (MARV), Rift Valley Fever virus (RVFV), Hantaviruses, and Dengue virus.

22. A nucleic acid molecule comprising a sequence encoding the polypeptide of claim 1.

23. A nucleic acid molecule of claim 22 further comprising an expression control sequence operably linked to the sequence encoding the polypeptide.

24. A recombinant cell comprising the nucleic acid molecule of claim 23.

25. A method of producing a polypeptide comprising culturing a recombinant cell of claim 24 under conditions suitable for expressing the encoded polypeptide and isolating the encoded polypeptide from the recombinant cells.


SEQUENCE LISTING (19 sequences)

SEQ ID NO: 1 - protein, 162 aa, Escherichia coli
MKTILPAVLFAAFATTSAWAAESVQPLEKIAPYPQAEKGMKRQVIQLTPQEDESTLKVELLIGQTLEVDCNLHRLGGKLENKTLEGWGYDYYVFDKVSSPVSTMMACPDGKKEKKFVTAYLGDAGMLRYNSKLPIVVYTPDNVDVKYRVWKAEEKIDNAVVR

SEQ ID NO: 2 - protein, 162 aa, Artificial Sequence
  variant polypeptides
MKTILPAVLFAAFATTSAWAAESVQPLEKIAPYPQAEKGMKRQVIQLTPQEDESTLKVELLIGQTLEVDCNLHRLGGKLENKTLEGWGYDYYVFDKVSSPVSTRMACPDGKKEKKFVTAYLGDAGMLRYNSKLPIVVYTPDNVDVKYRVWKAEEKIDNAVVR

SEQ ID NO: 3 - protein, 162 aa, Artificial Sequence
  variant polypeptides
MKTILPAVLFAAFATTSAWAAESVQPLEKIAPYPQAEKGMKRQVIQLTPQEDESTLKVELLIGQTLEVDCNLHRLGGKLENKTLEGWGYDYYVFDKVSSPRSTRMACPDGKKEKKFVTAYLGDAGMLRYNSKLPIVVYTPDNVDVKYRVWKAEEKIDNAVVR

SEQ ID NO: 4 - protein, 162 aa, Artificial Sequence
  variant polypeptides
MKTILPAVLFAAFATTSAWAAESVQPLEKIAPYPQAEKGMKRQVIQLTPQEDESTLKVELLIGQTLEVDCNLHRLGGKLENKTLEGWGYDYYVFDIVSSPVSTRMACPDGKKEKKFVTAYLGDAGMLRYNSKLPIVVYTPDNVDVKYRVWKAEEKIDNAVVR

SEQ ID NO: 5 - protein, 162 aa, Artificial Sequence
  variant polypeptides
MKTILPAVLFAAFATTSAWAAESVQPLEKIAPYPQAEKGMKRQVIQLTPQEDESTLKVELLIGQTLEVDCNLHDLGGKLENKTLEGWGYDYYVFDKVSSPVSTRMACPDGKKEKKFVTAYLGDAGMLRYNSKLPIVVYTPDNVDVKYRVWKAEEKIDNAVVR

SEQ ID NO: 6 - protein, 162 aa, Artificial Sequence
  variant polypeptides
MKTILPAVLFAAFATTSAWAAESVQPLEKIAPYPQAEKGMKRQVIQLTPQEDESTLKVELLIGQTLEVDCNLHRLGGKLENKTLEGWGYDYYVFDKVSSPRSTRMACPDGKKEKKFVTAYLGDAGMLRYNSMLPIVVYTPDNVDVKYRVWKAEEKIDNAVVR

SEQ ID NO: 7 - protein, 162 aa, Artificial Sequence
  variant polypeptides
MKTILPAVLFAAFATTSAWAAESVQPLEKIAPYPQAEKGMKRQVIQLTPQEDESTLKVELLIGQTLEVDCNLHRLGGKLENKTLEGWGYDYYVFDKVSSPVHTRMACPDGKKEKKFVTAYLGDAGMLRYNSKLPIVVYTPDNVDVKYRVWKAEEKIDNAVVR

SEQ ID NO: 8 - protein, 162 aa, Artificial Sequence
  variant polypeptides
MKTILPAVLFAAFATTSAWAAESVQPLEKIAPYPQAEKGMKRQVIQLTPQEDESTLKVELLIGQTLEVDCNLHRLGGKLENKTLEGWGYDYYVFDKVSSPVSTRMACPDGKKEKKFVTAYLGDAGMLRYNSMLPIVVYTPDNVDVKYRVWKAEEKIDNAVVR

SEQ ID NO: 9 - protein, 162 aa, Artificial Sequence
  variant polypeptides
MKTILPAVLFAAFATTSAWAAESVQPLEKIAPYPQAEKGMKRQVIQLTPQEDESTLKVELLIGQTLEVDCNLHRLGGKLENKTLEGWGYDYYVFDKVSSPGSTRMACPDGKKEKKFVTAYLGDAGMLRYNSMLPIVVYTPDNVDVKYRVWKAEEKIDNAVVR

SEQ ID NO: 10 - protein, 142 aa, Escherichia coli
AESVQPLEKIAPYPQAEKGMKRQVIQLTPQEDESTLKVELLIGQTLEVDCNLHRLGGKLENKTLEGWGYDYYVFDKVSSPVSTMMACPDGKKEKKFVTAYLGDAGMLRYNSKLPIVVYTPDNVDVKYRVWKAEEKIDNAVVR

SEQ ID NO: 11 - protein, 142 aa, Artificial Sequence
  variant polypeptides
AESVQPLEKIAPYPQAEKGMKRQVIQLTPQEDESTLKVELLIGQTLEVDCNLHRLGGKLENKTLEGWGYDYYVFDKVSSPVSTRMACPDGKKEKKFVTAYLGDAGMLRYNSKLPIVVYTPDNVDVKYRVWKAEEKIDNAVVR

SEQ ID NO: 12 - protein, 142 aa, Artificial Sequence
  variant polypeptides
AESVQPLEKIAPYPQAEKGMKRQVIQLTPQEDESTLKVELLIGQTLEVDCNLHRLGGKLENKTLEGWGYDYYVFDKVSSPRSTRMACPDGKKEKKFVTAYLGDAGMLRYNSKLPIVVYTPDNVDVKYRVWKAEEKIDNAVVR

SEQ ID NO: 13 - protein, 142 aa, Artificial Sequence
  variant polypeptides
AESVQPLEKIAPYPQAEKGMKRQVIQLTPQEDESTLKVELLIGQTLEVDCNLHRLGGKLENKTLEGWGYDYYVFDIVSSPVSTRMACPDGKKEKKFVTAYLGDAGMLRYNSKLPIVVYTPDNVDVKYRVWKAEEKIDNAVVR

SEQ ID NO: 14 - protein, 142 aa, Artificial Sequence
  variant polypeptides
AESVQPLEKIAPYPQAEKGMKRQVIQLTPQEDESTLKVELLIGQTLEVDCNLHDLGGKLENKTLEGWGYDYYVFDKVSSPVSTRMACPDGKKEKKFVTAYLGDAGMLRYNSKLPIVVYTPDNVDVKYRVWKAEEKIDNAVVR

SEQ ID NO: 15 - protein, 142 aa, Artificial Sequence
  variant polypeptides
AESVQPLEKIAPYPQAEKGMKRQVIQLTPQEDESTLKVELLIGQTLEVDCNLHRLGGKLENKTLEGWGYDYYVFDKVSSPRSTRMACPDGKKEKKFVTAYLGDAGMLRYNSMLPIVVYTPDNVDVKYRVWKAEEKIDNAVVR

SEQ ID NO: 16 - protein, 142 aa, Artificial Sequence
  variant polypeptides
AESVQPLEKIAPYPQAEKGMKRQVIQLTPQEDESTLKVELLIGQTLEVDCNLHRLGGKLENKTLEGWGYDYYVFDKVSSPVHTRMACPDGKKEKKFVTAYLGDAGMLRYNSKLPIVVYTPDNVDVKYRVWKAEEKIDNAVVR

SEQ ID NO: 17 - protein, 142 aa, Artificial Sequence
  variant polypeptides
AESVQPLEKIAPYPQAEKGMKRQVIQLTPQEDESTLKVELLIGQTLEVDCNLHRLGGKLENKTLEGWGYDYYVFDKVSSPVSTRMACPDGKKEKKFVTAYLGDAGMLRYNSMLPIVVYTPDNVDVKYRVWKAEEKIDNAVVR

SEQ ID NO: 18 - protein, 142 aa, Artificial Sequence
  variant polypeptides
AESVQPLEKIAPYPQAEKGMKRQVIQLTPQEDESTLKVELLIGQTLEVDCNLHRLGGKLENKTLEGWGYDYYVFDKVSSPGSTRMACPDGKKEKKFVTAYLGDAGMLRYNSMLPIVVYTPDNVDVKYRVWKAEEKIDNAVVR

SEQ ID NO: 19 - protein, 20 aa, Escherichia coli
MKTILPAVLFAAFATTSAWA